(19) United States
(12) Patent Application Publication
(10) Pub. No.: US 2002/0052010 A1
(43) Pub. Date: May 2, 2002

(54) Method of evaluation and selection of absorbent article

(76) Inventors: Takayuki Hisanaka, Kagawa-ken (JP); Yuri Imamura, Kagawa-ken (JP)

Correspondence Address: PAULA CAMPBELL EVANS, PALMER & DODGE LLP, One Beacon Street, Boston, MA, 02108, US

(21) Appl. No.: 09/888,289

(22) Filed: Jun. 22, 2001

(30) Foreign Application Priority Data
Sep. 1, 2000 (JP) 265695/2000

(51) Int. Cl. G01N 33/53; G01N 33/542; A61F 13/15

(52) U.S. Cl. 435/7.9; 604/358

(57) ABSTRACT

The present invention relates to a method of evaluating suitability of absorbent articles, and the method comprises using the immunoglobulin concentration in the body fluids as a measure. The method enables evaluating and selecting absorbent articles such as disposable diapers and sanitary napkins conforming to the user's physiological and psychological requirements.

### TECHNICAL FIELD

The present invention relates to a method of evaluating and selecting absorbent articles such as disposable diapers and sanitary napkins. More particularly, the present invention relates to a method of evaluating and selecting an absorbent article most appropriate for physiological and psychological requirements of users based on the immunoglobulin concentration in the users' body fluid.

### BACKGROUND ART

At present, absorbent articles such as disposable diapers, sanitary napkins, vaginal discharge liners, and breast milk pads are classified for sale in the market according to the dimensions (length, thickness, etc.), configuration, absorption amount, price, and the like.

For this reason, users select and purchase these absorbent articles according to the dimensions, configuration, absorption amount, price, and the like.

In addition, in the absence of expert sales personnel in shops, the purchasers have to select and purchase the articles displayed on the shelves according to the dimensions, configuration, absorption amount, and the like described on the packages.

For example, when buying a diaper for infants, the purchaser must select the articles according to the size or the body weight (e.g. the size for new-born babies, S, M, L, etc.) and the type classified according to the infant's ability to stand and walk or not (e.g. a type of assembling using tapes, or shorts type). When purchasing a diaper for adults, the waist size (S, M, L, etc.) and the form may be the measure for selection. Sanitary napkins are selected according to the amount of absorption and length (depending on the amount of vaginal discharge) and the form (with or without flaps).

Because the users usually purchase sanitary napkins themselves, they can select the articles most suited for their condition and favorite (tactile sense, form, absorption amount, etc.) through their experience. In addition, the sanitary napkin users can cope with troubles caused by the goods by themselves by receiving therapeutic measures in hospital, etc.

However, a purchaser is not usually the user in the case of diapers for infants or adults. Therefore, the goods are generally selected through the judgment of the purchaser, who is a care person or a helper, according to the size, leak-proof properties, etc. This tendency is particularly strong in the case of infants not possessing sufficient language and exercise capability or elderly persons with dementia symptoms.

Said selection is thus not necessarily in accord with the physiological or psychological requirements of the users. Selected goods may impart an unacceptable feeling of use (tactile sense, tightness, movability) and may result in externally induced diseases such as diaper rash (contact dermatitis) or may cause a psychic stress in the users. If such an absorbent article is continuously used, immune functions of the user may deteriorate due to a latent psychic stress.

Under such commercial circumstances, efforts of personnel involved in the development and manufacturing of absorbent articles may be directed to the purchaser's taste and convenience, and the development of product performance may not be in line with the user's requirements.

However, no method for correcting such a situation has conventionally been developed. There have been no appropriate measures used in the evaluation and selection of absorbent articles conforming to the user's comfort and tactile sensation.

### DISCLOSURE OF THE INVENTION

An object of the present invention is therefore to solve the above problems in the conventional technology. A specific object of the present invention is to provide a method of evaluating and selecting absorbent articles such as disposable diapers and sanitary napkins conforming to the user's physiological and psychological requirements. More specifically, an object of the present invention is to provide a method enabling developers and manufacturers of diapers to evaluate and select diapers closer to the user's physiological and psychological requirements such as wearing comfort and a method enabling purchasers to evaluate and select diapers more appropriate to the user's physiological and psychological requirements.

As a result of extensive studies with an objective of solving the above problems, the present inventors have found that the absorbent articles can be evaluated and selected by using the immunoglobulin concentration in the body fluids as a measure. This finding has led to the completion of the present invention.

In the investigation of various measures which can objectively estimate physiological and psychological conditions of the diaper users, the present inventors paid an attention to the immunoglobulin concentration and found that the degree of comfort of a diaper is related to the immunoglobulin concentration of the user's body fluids. Specifically, the inventors found that the immunoglobulin concentration of the body fluids of the user of an absorbent article when the user feels comfortable differs from the immunoglobulin concentration when he or she feels uncomfortable.

Therefore, the present invention relates to:

(1) A method of evaluating suitability of absorbent articles by using the immunoglobulin concentration in body fluids as a measure,

(2) A method of selecting an absorbent article suitable for a person wearing the article by using the immunoglobulin concentration in the wearer's body fluids as a measure,

(3) The method according to (1) or (2) above, comprising measuring the immunoglobulin concentration of the body fluids of a person wearing an absorbent article before and after urination to determine the change in the immunoglobulin concentration and using the rate of change as the measure.

(4) The method according to any one of (1) to (3) above, wherein the body fluid is saliva.

(5) The method according to any one of (1) to (4) above, wherein the immunoglobulin is secretory-type immunoglobulin A.

The present invention exhibits the special effect that an absorbent article satisfying physiological and psychological requirements of the wearer such as degree of comfort can be objectively and easily selected and evaluated by measuring the immunoglobulin concentration or the rate of change of the immunoglobulin concentration of the wearer's body fluids as a measure.

### DETAILED DESCRIPTION OF THE INVENTION AND PREFERRED EMBODIMENT

The present invention will be explained in more detail in the following description, which is not intended to be limiting of the present invention.

The absorbent articles in the present invention include disposable diapers, sanitary napkins, vaginal discharge liners, and breast milk pads. In addition, the present invention can also be applied to underwear shorts for incontinence and the like.

“Immune” or “immunity” as used with the term “immunoglobulin” and the like is a function of maintaining a biological homeostasis by preventing internal circumstances of the body from being disturbed by foreign matter such as bacteria invading the body from the external environment. Typical proteins involving immunity include lymphocytes, macrophages, blood neutrophils, and immunoglobulins.

Immunoglobulins are divided into five classes, IgD, IgG, IgE, IgA, and IgM, all of which can be used as the measure in the present invention.

IgA (immunoglobulin A) is a major immunoglobulin in external secretion fluid and usefully prevents the mucosal surface from being infected. IgA is contained in saliva, nasal mucus, discharge from the intestinal tract and air tubes in a large amount. IgA is also present in blood serum.

Blood samples are required for measurement of IgD, IgG, IgE, and IgM. On the other hand, secretory-type immunoglobulin (sIgA) can be determined by the analysis of saliva, the collection of which does not impart pain to the subject. Collection of blood should be avoided because the skin is damaged by injection with a needle which also imparts pain, which not only put stress on the subject, but also may induce a communicable disease from the wound.

From such a viewpoint, measurement of secretory-type immunoglobulin (sIgA) in saliva is particularly preferable in the present invention.

The secretory-type immunoglobulin A (sIgA) in saliva can be determined by any known analytical methods such as radioimmunoassay and the sandwich antibody method. One method of determining the concentration will hereinafter be described (see also “The Handbook of Human Factors and Ergonomics”, edited by The Japan Society of Physiological Anthropology, Instrumentation Research Department, Gihodoshuppan Co., Ltd., pages 430-432).

[Method of Collecting Saliva]

Five to six cotton balls, cut to a length of about 3 cm, are inserted into the back of the cheek or the like of the subject and left there for 5 minutes. The cotton balls which have absorbed saliva are placed in a centrifugal separator and centrifuged for 5 minutes at 3,000 rpm to separate and collect the saliva.

[Measurement of sIgA]

sIgA is determined using an EIA s-IgA test kit (manufactured by MBL Co., Ltd.) according to the following procedure.

1) Adaptation of Sample

(i) Saliva collected by centrifuge is diluted 20, 50, and 100 fold.

(ii) 0.4 ml of a reaction buffer solution is added to a test tube, followed by the addition of 10 μl of the diluted sample. The mixture is thoroughly mixed.

2) Primary Reaction

(i) An anti-human secretory component-binding polystyrene ball is added to the test tube.

(ii) The mixture is incubated at 37° C. for one hour.

3) Washing

(i) After the reaction, the reaction solution is removed by suction.

(ii) 1 ml of a phosphate buffer solution is added, shacked sufficiently, and removed by suction.

(iii) The steps (i) and (ii) are repeated twice.

4) Secondary Reaction

(i) After the above steps, 0.3 ml of an enzyme (peroxidase) labeled anti-human IgA is added to the test tube.

(ii) The mixture is allowed to stand at 20° C. for one hour.

5) Washing

The same procedure as the step (3) is repeated three times.

6) Enzyme Reaction

(i) 0.5 ml of an enzyme substrate (6-phenylenediamine) solution is added to a new test tube and then the polystyrene ball washed in the step 5) above is added.

(ii) The mixture is allowed to stand at 20° C. for 30 minutes.

7) Termination of Reaction

After the enzymatic reaction of the step 6) , 2 ml of 1 N sulfuric acid solution is added to the test tube to terminate the reaction.

8) Absorbance Measurement

The amount of 2,2′-diaminoazobenzole produced by the reaction is determined by measuring absorbance at 492 nm using a spectrophotometer. The value is applied to a standard curve to determine the sIgA concentration.

[Test of Wearing Absorbent Article]

The relation between the sIgA concentration thus determined and the sensation of use of the absorbent article is investigated.

(Absorbent Article)

The following two pants-type diapers are used as the absorbent articles.

TABLE 1

| | Rate of | Rewet | Absorbed | Retained |
|---|---|---|---|---|
| | absorption | amount | amount | water amount |
| | (sec) | (g) | (g/p) | (g/p) |
| Diaper I | 30.0 | 6.4 | 807.0 | 384.0 |
| Diaper II | 27.2 | 76.7 | 504.0 | 177.0 |

Note:

Rate of absorption: the time required for the total amount of 200 ml of a physiological saline solution added to the diaper is absorbed through the top sheet.

Rewet amount: the amount of a physiological saline solution absorbed in a filter paper placed on the top sheet of a diaper under a load of 35 g/cm² five minute after 200 ml of a physiological saline solution is added.

Absorbed amount: the value determined by subtracting the weight of a diaper before absorbing water from the weight of the diaper that was dipped in a physiological saline solution for 30 minutes, removed from the solution, and allowed to stand under a load of 35 g/cm² for 20 minutes to let water to escape. (g/p indicates the amount of water absorbed per one diaper.)

The amount of retained water: the value determined by subtracting the weight of a diaper before absorbing water from the weight of the diaper after absorbing water according to the above procedure of determining the amount of water absorption and removing the water by centrifugation at 150G for 90 seconds.

The diaper I is a sample representing a diaper exhibiting good performance in the rate of absorption, rewetting amount, amount of water absorption, and amount of retained water, whereas diaper II is a sample exhibiting poor performance in all of these tested items.

(Wearing Test Method)

Seven adult panelists A to G performed the wearing test. The panelists wore the diaper I or II in a room at 28° C. and 60% RH. After 25 minutes, simulated urine at 37° C. was applied between the diaper and the thigh using a silicon tube. The panelists continued to wear the diaper for 25 minutes. The sIgA concentration (ng/ml) before and immediately after discharge of simulated urine was determined for each panelist.

(Results)

The results are shown in Table 2.

TABLE 2

| | Diaper worn | | | | | |
|---|---|---|---|---|---|---|
| | Diaper I | | | Diaper II | | |
| | Before | After | | Before | After | |
| | uri- | uri- | Percentage | uri- | uri- | Percentage |
| | nation | nation | of change | nation | nation | of change |
| Panelist | ng/ml | ng/ml | (%) | ng/ml | ng/ml | (%) |
| A | 21.9 | 26.6 | 21.5 | 22.3 | 23.9 | 7.2 |
| B | 11.0 | 19.7 | 79.1 | 15.2 | 20.4 | 34.2 |
| C | 59.1 | 103.4 | 75.0 | 40.4 | 51.8 | 28.2 |
| D | 41.9 | 56.4 | 34.6 | 27.7 | 32.2 | 16.2 |
| E | 56.4 | 80.8 | 43.3 | 55.3 | 70.9 | 28.2 |
| F | 18.4 | 21.9 | 19.0 | 19.3 | 20.8 | 7.8 |
| G | 27.5 | 70.4 | 156.0 | 15.6 | 24.8 | 59.0 |
| Average | 33.7 | 54.2 | 61.2 | 28.0 | 35.0 | 25.8 |

As can be seen in Table 2, the diaper I with good performance exhibited a higher sIgA concentration after urination than the diaper II with poor performance. In addition, the diaper I exhibited a remarkable increase in sIgA concentration after urination, whereas the increase in the concentration after urination in the diaper II was small.

The Table 2 also shows the rate of the concentration change before and after urination. The average percentage of the change in the concentration after urination to before urination by seven panelists was 61.2% in the diaper I with good performance, whereas the percentage was 25.8% in the diaper II with poor performance.

As a result, the diaper I with good performance was confirmed to exhibit a high concentration after urination and a significantly high percentage of the concentration change after urination. This indicates that the appropriateness of a diaper can be judged by determining the sIgA concentration after urination and/or the percentage of the sIgA concentration change after urination. More specifically, a diaper exhibiting a high sIgA concentration after urination and/or a high percentage of the sIgA concentration change after urination can be judged to be a good diaper.

These results indicate that the sIgA concentration and its rate of change are not only applicable to evaluation of diapers in the development and manufacture, but also usable as a measure for selecting a diaper imparting a comfortable feeling to users such as infants and aged persons who cannot express their own feelings.

If these results are applied, the present invention exhibits excellent effect that it is possible to provide diapers more appropriate to the users (wearers) through developing and manufacturing diapers exhibiting a high sIgA concentration after urination and a high percentage of the concentration change after urination.

In addition, since the sIgA concentration of the wearer can be increased by wearing the diaper I rather than the diaper II, continued use of the diaper selected by this method of evaluation is expected to be able to increase the wearer's immunological competence.

In the above description, the present invention has been explained referring to the relationship between the absorption performance of a diaper and the immunoglobulin concentration. An absorbent article most appropriate for the user in terms of other properties such as tactile sense, tightness, and movability can also be selected by determining immunological conditions in the same manner.

In addition, although the relationship between the absorbent article and immunoglobulin concentration is determined using a diaper as an example of the absorbent article in the above description, the same results can also be expected for other absorbent articles.

### EXAMPLES

The present invention will now be described by way of examples, which should not be construed as limiting the present invention.

Three types of diapers I-III with different absorption performance (see Table 3) were worn by seven panelists.

The panelists wore the diapers in a room at 28° C. and 60% RH. After 25 minutes, simulated urine at 37° C. was discharged between the diaper and the thigh through a silicon tube. The panelists continued to wear the diaper for 25 minutes.

[sIgA Concentration]

The sIgA concentration (ng/ml) before and immediately after discharge of simulated urine was determined for each panelist. The average sIgA concentration of the seven panelists and the concentration change (%) before and after urination are shown in Table 4.

[Degree of Comfort]

Degree of comfort felt by the wearers after urination was evaluated according to the following criteria of three levels.

3: Almost no change in the wearing sensation after urination as compared with before urination.

2: The wearing sensation was slightly more uncomfortable after urination as compared with before urination.

1: The wearing sensation was uncomfortable after urination as compared with before urination.

The values averaged for the seven panelists are shown in Table 4.

[Observation of Skin Conditions]

Aged persons requiring care wore the diapers, which were replaced seven times a day on average. After five days, the skin conditions were observed by the naked eye. The results are also shown in Table 4.

TABLE 3

| | Rate of | Rewet | Absorbed | Retained |
|---|---|---|---|---|
| | absorption | amount | amount | water amount |
| | (sec) | (g) | (g/p) | (g/p) |
| Diaper I | 30.0 | 6.4 | 807.0 | 384.0 |
| Diaper II | 27.2 | 76.7 | 504.0 | 177.0 |
| Diaper III | 28.6 | 43.6 | 638.0 | 256.0 |

TABLE 4

| | s-IgA | | | | |
|---|---|---|---|---|---|
| | Before | After | Concentration | Degree | |
| | urination | urination | change | of | Condition of |
| | (a) | (b) | (%) | comfort | the skin |
| Diaper | 33.7 | 54.2 | 61.2 | 2.7 | No abnor- |
| I | | | | | malities |
| Diaper | 28.0 | 35.0 | 25.8 | 1.1 | erythema |
| II | | | | | |
| Diaper | 27.8 | 38.9 | 39.9 | 1.9 | Slight |
| III | | | | | erythema |

Based on the results of Tables 3 and 4, the concentration after urination and the change in the concentration before and after urination have been confirmed to be proportionate to degree of comfort of the wearers. In addition, diapers exhibiting a high sIgA concentration after urination and a high percentage in the concentration change after urination have been confirmed to be more comfortable on the skin of the wearers.

### INDUSTRIAL USEFULNESS

The use of immunoglobulin as a measure of evaluation according to the present invention ensures objective evaluation of an absorbent article possessing appropriate functions. The present invention therefore can be applied to development of an absorbent article possessing appropriate functions. In addition, an absorbent article satisfying the wearer's desired degree of comfort can be selected and purchased by determining the wearer's concentration and the change in the concentration after urination.

1. A method of evaluating suitability of absorbent articles comprises using the immunoglobulin concentration in the body fluids as a measure.

2. A method of selecting an absorbent article suitable for a person wearing the article comprises using the immunoglobulin concentration in the wearer's body fluids as a measure.

3. The method according to claim 1 or 2, comprising measuring the immunoglobulin concentration of the body fluids of a person wearing an absorbent article before and after urination to determine the change in the immunoglobulin concentration and using the rate of the change as the measure.

4. The method according to any one of claims 1 to 3, wherein the body fluid is saliva.

5. The method according to any one of claims 1 to 4, wherein the immunoglobulin is secretory-type immunoglobulin A.
